(19) United States
(12) Patent Application Publication
(10) Pub. No.: US 2008/0161817 A1
(43) Pub. Date: Jul. 3, 2008

(54) Intervertebral distraction device

(75) Inventors: Matthew L. Parsons, Dartmouth, MA (US); Domagoj Coric, Charlotte, NC (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: DePuy Spine, Inc., Raynham, MA (US)

(21) Appl. No.: 11/528,747

(22) Filed: Sep. 28, 2006

(51) Int. Cl. A61B 17/58

(52) U.S. Cl. 606/90

(57) ABSTRACT

An intervertebral distraction instrument comprises a first vertebra engaging member and a second vertebra engaging member. The first vertebra engaging member includes a vertebra anchor member configured to prevent lateral movement of the distraction instrument while allowing anterior-posterior movement of the distraction instrument during engagement with the first vertebrae. A similar anchor member may also be provided on the second vertebra engaging member. The anchor member of the distraction instrument may be provided on the end portion of an elongated arm of the distraction instrument. In one embodiment, the anchor member of the distraction instrument comprises a keel that includes edge configured to cut into the vertebra. In another embodiment, the anchor member of the distraction instrument comprises a spike.

### FIELD

This invention relates to the field of prosthetics, and more particularly, to an intervertebral distraction instrument designed to separate vertebrae during an intervertebral implant procedure.

### BACKGROUND

The human spine consists of twenty-four small bones known as vertebrae, or “vertebral bodies,” that protect the spinal cord and provide stability to the torso. The vertebrae are arranged in a column and stacked vertically upon each other. Between each vertebra is a fibrous bundle of tissue called an intervertebral disc. These intervertebral discs act as a cushion to the spinal column by absorbing energy and transmitting loads associated with everyday movement. They also prevent the vertebrae from rubbing against each other.

Each intervertebral disc comprises two distinct regions. A firm outer region, the annulus, maintains the shape of the intervertebral disc. An inner region, the nucleus, provides a resilient tissue that enables the disc to function as a shock absorber. Over time, the normal aging process causes the intervertebral discs to degenerate, diminishing their water content and thereby reducing their ability to properly absorb the impact associated with spinal movements. Diminished water content in the intervertebral discs may also cause the vertebrae to move closer together. Tears and scar tissue can weaken the discs, resulting in injury. When the discs wear out or are otherwise injured, they may cause pain and limit activity.

Pain and limited activity from injured intervertebral discs can potentially be relieved by a surgical procedure called artificial disc replacement. In this procedure, the damaged intervertebral disc is replaced by a prosthetic disc. This disc prosthesis is generally comprised of two endplates and a center core. The endplates include fixation elements on their outer surface in the form of either fins (also referred to as “keels”) or spikes (also referred to as “teeth”). The fixation elements are provided to secure the disc prosthesis to the vertebrae. Keels have the advantage of assuring implant alignment with the chisel cut that is made in the vertebra for the keel. Therefore, once the surgeon cuts the slot for the keel in the vertebral body, he or she can be assured that after the keel is inserted in the slot, the implant will not drift away from that position. With a spiked or “toothed” implant, the surgeon can not be completely sure that the bony anatomy of the vertebra will not redirect or laterally shift the implant during the insertion process.

During a typical artificial disc replacement procedure, the damaged disc is first removed via an anterior surgical approach and the end surfaces of the two exposed vertebrae are cleared of debris. A distraction instrument is then used to spread the vertebrae apart. With the vertebrae spread apart, the artificial disc is slid in place between the vertebrae.

In one procedure, the distraction instrument also serves as an installation instrument. In particular, in addition to being configured to spread apart the two vertebrae, the instrument is also configured to slide the artificial disc into place while the vertebrae remain separated. A central ramp is provided on the instrument to facilitate sliding of the implant between the vertebrae. With a toothed implant, it is important that the distraction instrument remains in place and does not shift laterally during the implantation procedure. Any lateral shifting of the distraction instrument during the implantation procedure could result in improper placement of the toothed implant. However, prior art distraction instruments typically do not include features designed to limit lateral shifting of the distraction instrument.

Accordingly, it would be advantageous to provide a distraction instrument that includes features to prevent the distraction instrument from moving laterally during the implantation procedure. It would also be advantageous if the same features of the distraction instrument allowed anterior-posterior movement during the implantation procedure. It would be further advantageous if such features could be easily incorporated into existing distraction instrument designs.

### SUMMARY

An intervertebral distraction instrument comprises a first vertebra engaging member and a second vertebra engaging member. The first vertebra engaging member includes a vertebra anchor member configured to prevent lateral movement of the distraction instrument while allowing anterior-posterior movement of the distraction instrument during engagement with the first vertebrae. A similar anchor member may also be provided on the second vertebra engaging member.

The anchor member of the distraction instrument may be provided on the end portion of an elongated arm of the distraction instrument. In one embodiment, the anchor member of the distraction instrument comprises a keel (also referred to herein as a fin). The keel may be any of various shapes, such as, for example, ramped, spiked, pie-slice shaped, or of a constant cross-section along an axis extending from its distal end to its proximal end. The keel may include at least one sharp edge configured to cut into the first vertebra. Alternatively, the keel may be placed into a previously cut slot in the vertebra.

In another embodiment, the anchor member of the distraction instrument comprises a spike with a sharp tip. The spike may be provided as any of numerous different shapes, such as cone shaped or pie-slice shaped. In one embodiment the spike is provided on the elongated arm of the distraction member and extends outward from an end portion of the elongated arm.

The above described features and advantages, as well as others, will become more readily apparent to those of ordinary skill in the art by reference to the following detailed description and accompanying drawings.

### BRIEF DESCRIPTION OF THE DRAWINGS

FIG. 1 shows a perspective view of an intervertebral distraction instrument holding an implant in a retracted position;

FIG. 2 shows a side view of the end portions of two elongated arms of the intervertebral distraction instrument of FIG. 1 with a vertebra anchor provided on each of the end portions;

FIG. 3 shows a side view of the end portions of the intervertebral distraction instrument of FIG. 2 with the bone anchors engaging the vertebrae;

FIG. 4 shows a perspective view of an alternative embodiment of the vertebra anchor of FIG. 2;

FIG. 5A shows a perspective view of another alternative embodiment of the vertebra anchor of FIG. 2; and

FIG. 5B shows a top view of the vertebra anchor of FIG. 5A;

FIG. 6 shows a side view of yet another alternative embodiment of the vertebra anchor of FIG. 2; and

FIG. 7 shows a perspective view of another alternative embodiment of the vertebra anchor of FIG. 2.

### DESCRIPTION

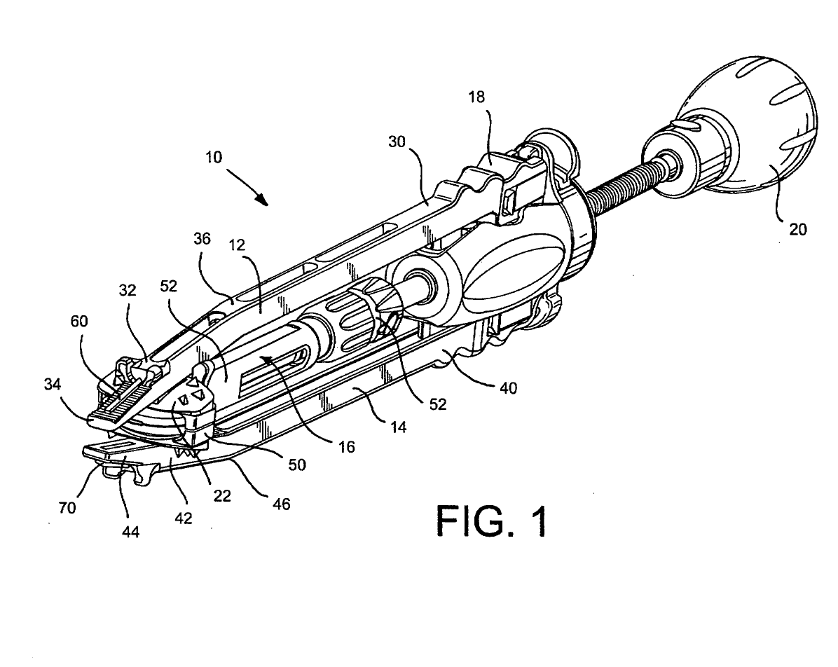

With reference to FIG. 1, an intervertebral distraction instrument 10 comprises a first vertebra engaging member 12, a second vertebra engaging member 14, and a prosthesis insertion member 16. A handle 18 is provided on the instrument with a knob 20 that provides for movement of the prosthesis insertion member toward the end of the first and second vertebra engaging members. The intervertebral distraction instrument 10 is designed to facilitate insertion of a prosthetic device 22 held by the prosthesis insertion member 16 into an intervertebral space between two adjacent vertebra.

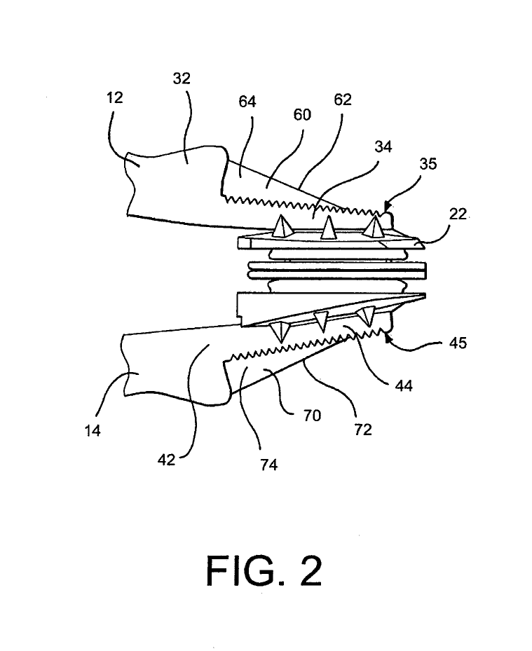

In the embodiment of FIG. 1, the first vertebra engaging member 12 is provided as an upper elongated distraction arm. The upper elongated arm 12 includes a proximal end 30 and a distal end 32. A finger 34 extends from the distal end 32 of the arm 12. The finger 34 is a thin rectangular tab with a vertebra engaging surface 35 (see FIG. 2) on the upper portion of the tab.

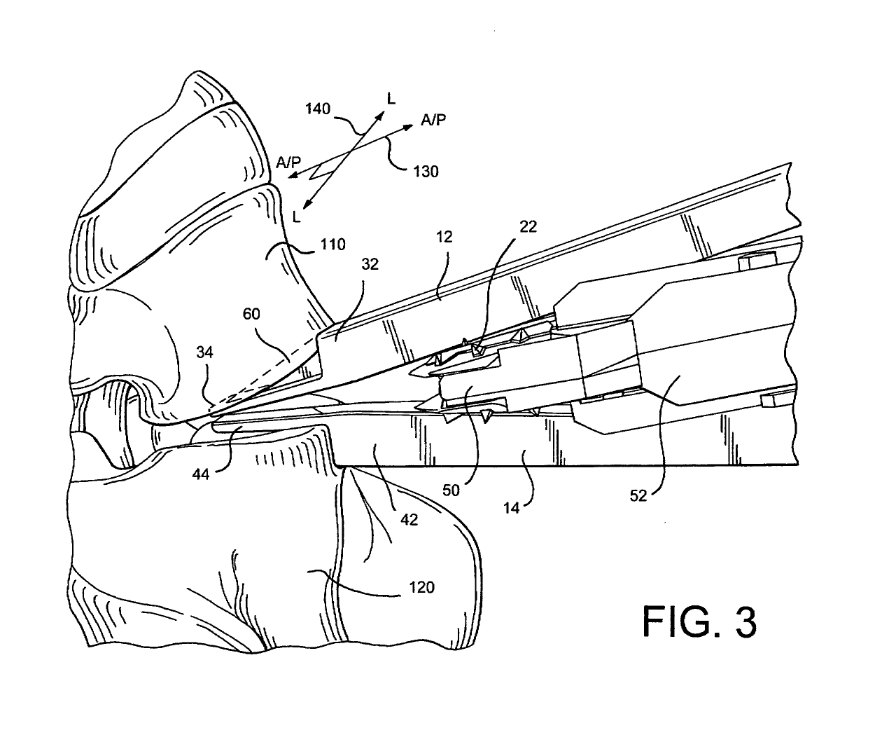

The second vertebra engaging member 14 is provided as a lower elongated distraction arm, and is generally symmetric to the upper elongated arm 12. Accordingly, the lower elongated arm 14 includes a proximal end 40, a distal end 42, and a finger 44 extending from the distal end 42. The finger 44 includes a vertebra engaging surface 45 on the lower portion of the finger 44 (see FIG. 3). Both the upper elongated arm 12 and the lower elongated arm 14 include a bend 36, 46 near the center such that the distal ends 32, 42 of the arms come together at the fingers 34, 44.

The prosthesis insertion member 16 is situated between the upper elongated arm 12 and the lower elongated arm 14. The prosthesis insertion member 16 includes a prosthesis holding member in the form of a grip 50 and a prosthesis insertion arm 52. The prosthesis grip 50 is designed to grasp or release the prosthetic device 22. The prosthesis insertion arm 52 is designed to move relative to the elongated arms 12, 14, and thus move the prosthetic device 22 retained by the grip 50 toward the distal ends 32, 42 of the elongated arms 12, 14.

The handle 18 is provided at the proximal ends 30, 40 of the elongated arms 12, 14. Rotational movement of the knob 20 relative to the handle 18, such as defined by a threaded mechanism, results in axial movement of the prosthesis insertion arm 52 and grip 50 relative to the elongated arms 12, 14. When the insertion arm 52 and grip 50 move in the axial direction, the prosthetic device 22 held within the grip 50 moves in the axial direction toward the distal ends 34, 44 of the elongated arms 12, 14. As the prosthesis device moves between the fingers 34, 44, the elongated arms 12, 14 are forced apart, thereby distracting the adjacent vertebrae and opening the disc space to make way for the prosthetic device 22.

FIG. 2 shows a side view of the prosthetic device positioned at the end of the elongated arms 12, 14 and positioned between the fingers 34, 44. As best seen in FIG. 2, a first vertebra anchor member 60 is provided as a keel on the distal end of the first elongated arm 12. In the embodiment of FIG. 2, the vertebra anchor member 60 is generally pyramidal in shape and includes a sharp upper edge 62, and two triangular faces 64. The sharp upper edge 62 of the anchor member 60 is ramped such that the height of the upper edge 62 increases moving toward the proximal end of the elongated arm 12. The sharp upper edge 62 provides a blade and is configured to cut into the vertebra or easily enter a groove already formed in the vertebra. Two triangular faces 64 extend down from the upper edge 62. However, only one of the triangular faces 64 can be seen in FIG. 2. Both of the triangular faces 64 are generally smooth, allowing the faces to cut into the vertebra.

A second vertebra anchor member 70 is provided as a keel on the lower elongated arm 14. The second vertebra anchor member 70 is generally symmetrical to the first vertebra anchor member 60. Accordingly, the second vertebra anchor member 70 is pyramidal in shape and includes a sharp lower edge 72 and two faces 74 extending from the edge 72.

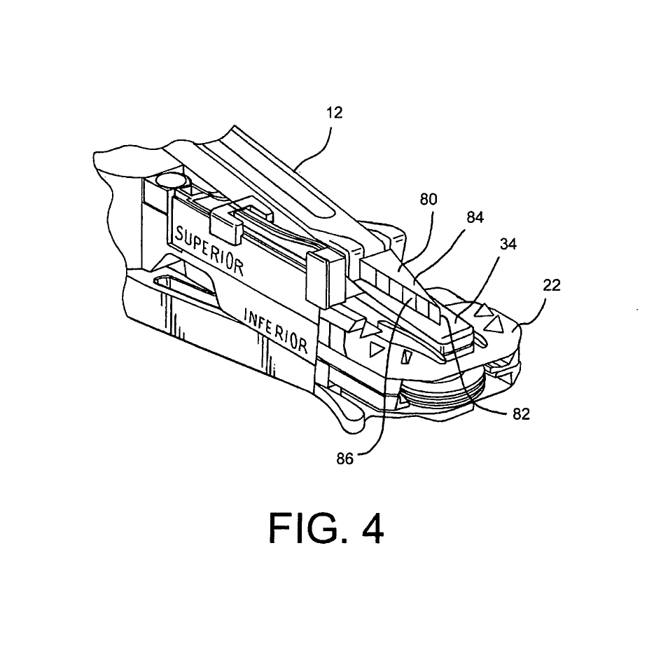

One of skill in the art will recognize that although the anchor members 60, 70 have been shown as pyramidal in the embodiment of FIG. 3, other shapes of anchor members are possible. For example, the anchor members 60, 70 may be relatively thin planar blades that ramp out from the fingers 34, 44 rather than the pyramidal configuration of FIG. 2. Other examples of alternative anchor members are shown in FIGS. 4-7.

In the embodiment of FIG. 4, the anchor member 80 is shown as a pie-slice shaped fin. The anchor member 80 of FIG. 4 includes a sharp distal edge 82, and an upper face 84 that extends proximally from the sharp edge 82. Two rectangular side faces 84 also extend from the sharp edge. The anchor member 80 is designed to cut into a solid vertebra or be inserted into a groove in a vertebra with the sharp edge 82 leading the insertion of the anchor member.

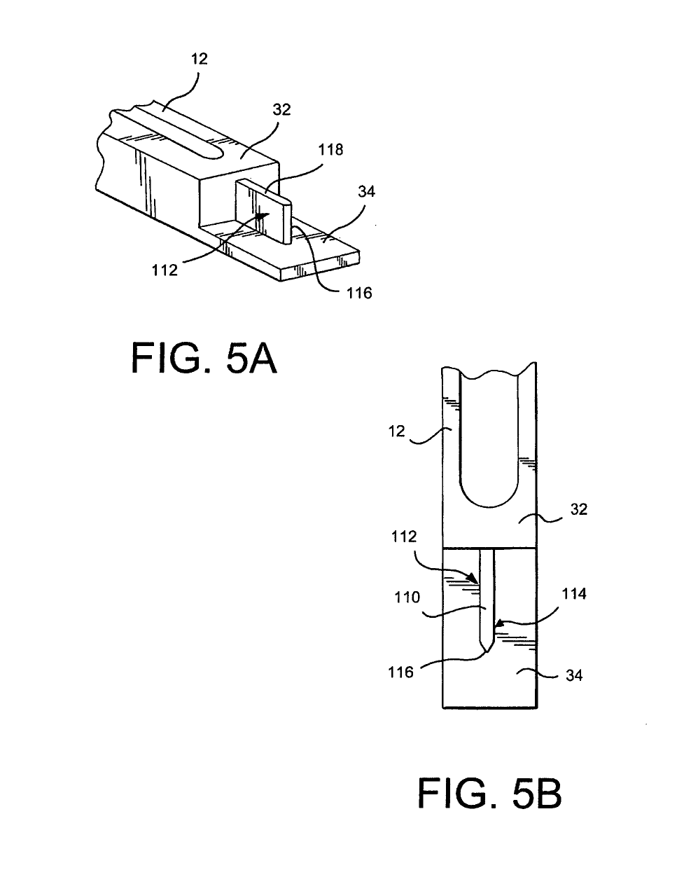

The embodiment of FIGS. 5A and 5B is similar to the embodiment of FIG. 4 in that the anchor member 110 is connected to the finger 34, but the anchor member has a different shape. In FIGS. 5A and 5B, the anchor member 110 is provided as a fin on the surface of the finger 34. The fin 110 includes two parallel opposing rectangular surfaces 112, 114 that extend perpendicularly from the tab 34. The rectangular surfaces 112, 114 taper into a sharp leading edge 116. A relatively blunt upper surface 118 is provided at the top of the fin 110. Because the surfaces 112 and 114 are parallel, the cross-sectional size and shape of the fin is generally constant apart from the tapered portion leading to the edge 116.

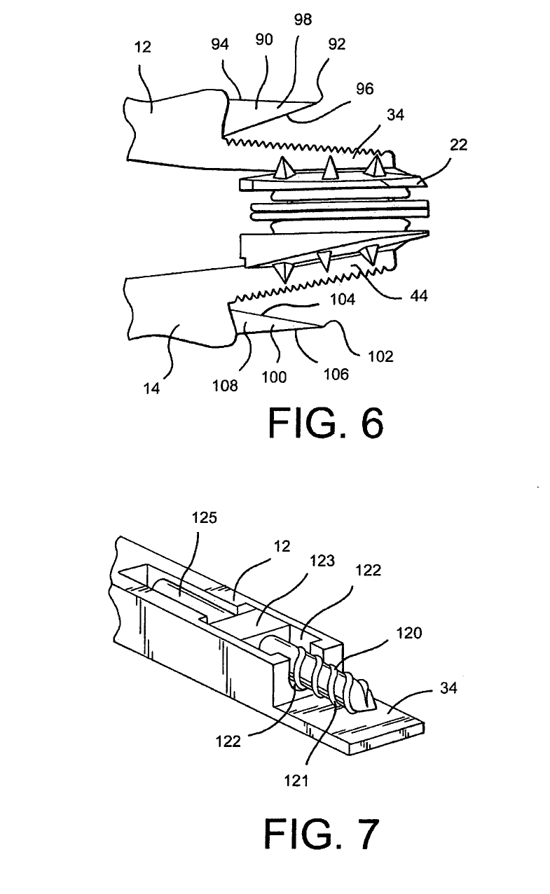

In the embodiment of FIG. 6, a spike-shaped anchor member 90 extends from the elongated upper arm 12 above the finger 34. The spike-shaped anchor member 90 includes a sharp distal tip 92. In the embodiment of FIG. 6, the spike-shaped anchor member also includes two edges 94, 96. One or both edges 94, 96 may be sharp edges. The spike 90 also includes two opposing faces 98, but only one of the faces is seen in FIG. 6. A similar anchor member 100 extends from the elongated lower arm 14, and is separate from the finger 44. Accordingly, the anchor member 100 includes a sharp distal tip 102, two edges 104, 106, and two opposing faces 108. In alternative embodiments, the spike anchor members of FIG. 6 may be shaped differently. For example, the anchor members 90, 100 may be conical in shape with a sharp tip. As another example, the anchor members 90, 100 may be pyramidal in shape, similar to the anchor members 60, 70 of FIG. 2, but separate from the fingers 34, 44.

The embodiment of FIG. 7 is similar to the embodiment of FIG. 6 in that the anchor member is separate from the finger 34. However, in the embodiment of FIG. 7, the spike-shaped anchor member is provided as a Caspar pin 120 that is embedded into a pocket 122 on the elongated arm 12. The Caspar pin 120 includes a threaded shaft 121 a head 123, and a rear post 125. The head 123 and rear post 125 are configured to slide within the pocket 122 in the axial direction of the pin with the threaded shaft 121 extending from the pocket 122 above the finger 34. The Caspar pin 120 is removable from the pocket 122 and may be replaced when the pin becomes dull or damaged. This provides the potential advantage of being able to insert and position one or more anchor members prior to inserting the entire distraction instrument 10 into the surgical site. The Caspar pin 120 and pocket 122 are arranged with a locking mechanism allowing the pin 120 to be locked in place upon the instrument 10 during use, and later unlocked and removed from the pocket 122. The geometry of the locking mechanism (e.g. the length of the pocket) allows for anterior/posterior translation of the pin 120 in the pocket 122, but does not allow lateral motion of the pin relative to the pocket.

In operation, the intervertebral distraction instrument 10 described herein may be used to separate vertebrae and insert an implant while limiting lateral movement of the instrument 10, but still allowing anterior-posterior movement of the instrument 10. With reference again to FIG. 1 an intervertebral prosthesis 22 is shown positioned within the intervertebral distraction instrument 10. Rotation of the knob 20 relative to the handle 18 causes the insertion arm 52, grip 50 and prosthesis 22 to move toward the distal end 32.

As shown in FIG. 3, once the damaged disc is removed from the intervertebral space, the distal ends 32, 34 of the elongated arms 12, 14, engage the vertebrae 110, 120. Blunt edges of the elongated arms are positioned against the vertebral bodies 110, 120, and the fingers 34, 36 are inserted into the intervertebral space. When the fingers 34, 36 are inserted into the intervertebral space, the vertebra anchors 60, 70 cut into the vertebra as the distraction instrument moves in the anterior-posterior direction. Alternatively, the vertebra anchors 60, 70 may be positioned into previously cut channels in the vertebra.

As the prosthesis 22 is gradually moved toward the distal ends 32, 42 of the elongated arms 12, 14, and the intervertebral space, the prosthesis 22 causes the elongated arms 12, 14 to spread apart. As the elongated arms 12, 14 and fingers 34, 44 are moved apart, space is created between the vertebral bodies 110, 120 for the prosthesis 22. Depending upon the configuration of the vertebra anchors 60, 70, separation of the fingers 34, 44 may cause the vertebra anchors 60, 70 to cut further into the vertebra 110, 120. In FIG. 3, the finger 34 and vertebra anchor 60 is shown in part by dotted lines in order to show the engagement of the vertebra anchor with the vertebra 110. The surgeon may pre-cut a groove in the vertebra 110 to receive the vertebra anchor 60, or a sharp blade or tip on the vertebra anchor 60 may cut into the vertebra without a pre-cut groove. In either case, the configuration of the vertebra anchor 60 and its engagement with the groove cut in the vertebra is such that movement of the vertebra anchor 60 in the anterior/posterior direction (indicated by line 130 in FIG. 3) is allowed, but movement in the lateral direction (indicated by line 140 in FIG. 3) is limited. Of course, this limited movement is recognized primarily at the distal end of the distraction instrument, and the surgeon should remain careful about laterally pivoting the proximate end of the instrument about the more stationary distal end. Because the vertebra anchors 60, 70 substantially prevent motion in the lateral direction, insertion of a prosthesis 22 with teeth as the fixation members is facilitated and proper placement of such a prosthesis 22 is more easily achieved.

Although the present invention has been described with respect to certain preferred embodiments, it will be appreciated by those of skill in the art that other implementations and adaptations are possible. For example, the vertebra anchor may take on different configurations, shapes and arrangements from those disclosed herein. As another example, the vertebra anchor may be detachable from the elongated arm, such that the vertebra anchor can be inserted independently (as a spike, nail, screw, or staple, etc.), and the elongated arm could attach to the anchor as the arm is initially positioned in the intervertebral space. In addition to other embodiments, there are advantages to individual advancements described herein that may be obtained without incorporating other aspects described above. Therefore, the spirit and scope of the appended claims should not be limited to the description of the preferred embodiments contained herein.

What is claimed is

1. An intervertebral distraction instrument configured to separate a first vertebra and a second vertebra, the intervertebral distraction instrument comprising:
a) a first vertebra engaging member, the first vertebra engaging member including a vertebra anchor member configured to prevent lateral movement of the distraction instrument and allow anterior-posterior movement of the distraction instrument during engagement with the first vertebra; and
b) a second vertebra engaging member configured to move relative to the first vertebra engaging member.

2. The intervertebral distraction instrument of claim 1 wherein the vertebra anchor member comprises a keel.

3. The intervertebral distraction instrument of claim 2 wherein the keel is ramped.

4. The intervertebral distraction instrument of claim 1 wherein the vertebra anchor member includes a sharp edge configured to cut into the first vertebra.

5. The intervertebral distraction instrument of claim 1 wherein the vertebra anchor member comprises a spike.

6. The intervertebral distraction instrument of claim 5 wherein the spike is cone shaped.

7. The intervertebral distraction instrument of claim 5 wherein the spike is pie-slice shaped.

8. The intervertebral distraction instrument of claim 1 wherein the first vertebra engaging member comprises a first elongated arm and the second vertebra engaging member comprises a second elongated arm.

9. The intervertebral distraction instrument of claim 8 wherein the vertebra anchor member is removeably connected to the first elongated arm.

10. The intervertebral distraction instrument of claim 9 wherein the vertebra anchor member comprises a pin partially retained within a pocket on the first elongated arm.

11. The intervertebral distraction instrument of claim 8 wherein the first vertebra engaging member further comprises an end tab extending from the first elongated arm.

12. The intervertebral distraction instrument of claim 11 wherein the vertebra anchor of the first vertebra engaging member comprises a spike that extends outward from the first elongated arm.

13. The intervertebral distraction instrument of claim 8 further comprising an implant holding member positioned between the first elongated arm and the second elongated arm.

14. The intervertebral distraction instrument of claim 13 further comprising a knob, wherein movement of the knob forces the implant holding member toward the end of the first elongated arm and the second elongated arm, resulting in movement of the first arm relative to the second arm.

15. The intervertebral distraction instrument of claim 1 wherein the second vertebra engaging member includes a second vertebra anchor member configured to prevent lateral movement of the distraction instrument and allow anterior-posterior movement of the distraction instrument during engagement with the second vertebra.

16. The intervertebral distraction instrument of claim 1 wherein movement of the second vertebra engaging member relative to the first vertebra engaging member is designed to separate the first vertebra from the second vertebra

17. An intervertebral distraction instrument configured to engage a first vertebra and a second vertebra, the intervertebral distraction instrument comprising:
a) a first distraction arm with a first vertebra engaging surface provided on the end of the first distraction arm;
b) a second distraction arm with a second vertebra engaging surface provided on the end of the second distraction arm, the second distraction arm configured to move relative to the first distraction arm; and
c) a vertebra anchor member provided on the first vertebra engaging surface, the vertebra anchor member configured to prevent lateral movement of the distraction instrument and allow anterior-posterior movement of the distraction instrument during engagement with the first vertebrae.

18. The intervertebral distraction instrument of claim 17 further comprising a second vertebra anchor member provided on the second vertebra engaging surface, the second vertebra engaging member configured to prevent lateral movement of the distraction instrument and allow anterior-posterior movement of the distraction instrument during engagement with the second vertebra.

19. The intervertebral distraction instrument of claim 17 wherein the vertebra anchor member includes a sharp edge configured to cut into the first vertebra.

20. The intervertebral distraction instrument of claim 17 wherein the vertebra anchor member includes a sharp tip configured to cut into the first vertebra.

21. An intervertebral distraction instrument configured to engage a first vertebra and a second vertebra, the intervertebral distraction instrument comprising:
a) a first vertebra engaging member, the first vertebra engaging member including means for preventing lateral movement of the distraction instrument while allowing anterior-posterior movement of the distraction instrument during engagement of the first vertebra engaging member with the first vertebrae; and
b) a second vertebra engaging member configured to move relative to the first vertebra engaging member.

22. A method of inserting an intervertebral prosthesis, the method comprising the steps of:
a) providing an intervertebral distraction instrument comprising a vertebra anchor configured to prevent lateral movement of the distraction instrument during engagement with the first vertebra;
b) engaging the vertebra anchor with the vertebra such that it cuts into the vertebra; and
c) inserting the prosthesis in an intervertebral space during engagement of the vertebra anchor with the vertebra.

23. The method of claim 22 further comprising the step of cutting a groove in the vertebra before the step of engaging the vertebra anchor with the vertebra such that it cuts into the vertebra.
